(19) United States
(12) Patent Application Publication
(10) Pub. No.: US 2025/0017765 A1
(43) Pub. Date: Jan. 16, 2025

(54) COOLING THERAPY APPARATUS

(71) Applicant: Nice Recovery Systems LLC, Boulder, CO (US)

(72) Inventors: Michael Ross, Boulder, CO (US); Joel Meisinger, Boulder, CO (US); Gerry Taylor, Boulder, CO (US)

(21) Appl. No.: 18/221,237

(22) Filed: Jul. 12, 2023

(51) Int. Cl. A61F 7/00; A63B 22/06

(52) U.S. Cl. CPC A61F 7/007; A63B 22/0664; A61F 2007/0036; A61F 2007/0075; A61F 2007/0087; A63B 2225/66

(57) ABSTRACT

An apparatus is described that provides cooling therapy for a user of the apparatus. A user may place his or her palm(s) of the hand(s) on or close to the present invention to cool the palm(s) and thereby reduce heat in the user's body. The invention may include one or more internal fans pulling ambient air in through multiple air inlets and discharging cooled air from multiple outlets as the user holds the device of the present invention in his or her hand(s). Air circulation within the device, through a heat exchanger and/or thermoelectric module in the device, such as a Peltier cooling module, promotes cooling of the device and objects (such as the user's hands holding the device) in close proximity to the device. As a user's palms are cooled over a time period of use of the device of the present invention, the user's body temperature is also cooled, which reduces negative impacts of body heat on the user's muscles, and thereby enhances physical performance by the user for an added period of time.

### CROSS-REFERENCE TO RELATED APPLICATIONS

This application makes no priority claim.

### TECHNICAL FIELD

The present invention relates generally to a device for cooling therapy, and more particularly to a hand-held device for cooling the palms of a user's hands to thereby improve performance of a physical endeavor, such as physical exercise. In one example embodiment, a thermally conductive device is configured with a plurality of air inlets and outlets adapted to permit one or more fans to circulate air within the device for cooling objects (e.g., a user's palms) in close proximity thereto. In the aforementioned example embodiment, the device is configured to be hand-held, and a user may place one or both of the user's palms on the device to cool the body temperature of the user.

### BACKGROUND AND SUMMARY OF THE INVENTION

Muscle strength, endurance, performance and the like may be adversely impacted by the accumulation of internal heat in body tissues during and/or after exercise. By way of example and not limitation, it is known that performance of exercises may result in significant accumulation of internal heat in body tissues, temporarily reducing muscle performance and endurance. Although excess internal heat in body tissues may decrease after an extended amount of time has passed following the completion of exercise(s), it may be preferable to remove excess internal heat from the body sooner. By way of example and not limitation, in a situation where one desires to engage in continued and/or subsequent exercises, and significant internal heat in body tissues has accumulated, muscle strength, endurance, performance and the like may be improved to promote said continued and/or subsequent exercises by cooling the body.

It is known that heat may be extracted from the body by transferring heat away from certain skin surfaces substantially free of hair. As a specific example, on the surface of one's hands (as well as the bottom of one's feet and one's upper face), glabrous skin is present, and temperature regulation of glabrous skin is believed to affect core body temperature. A known device for extracting body heat in an attempt to, e.g., improve muscle strength, endurance, performance and the like, includes a cooling mitten having cool water pathways adapted to circulate cool water to substantially hairless skin of the palms, soles and/or face to cool blood at surfaces thereof. The aforementioned skin may comprise relatively high volumes of blood, and the known device may include a vacuum for increasing blood volume at said surfaces. Disadvantages of the known device include by way of example and not limitation, that it is highly complex, requires a large amount of storage and operation space, is expensive to end users, and the effects of using a vacuum may not be predictable for different users. The necessity for fluid pathways and pressure regulation requires the known device to provide substantial space and material to permit said fluid pathways and pressure regulation. Another known device for extracting body heat in an attempt to, e.g., improve muscle strength, endurance, performance and the like, includes a metallic bar filled with a fluid such as water and preferably refrigerated to provide a cool surface to contact skin. Disadvantages of the known device include that temperature of the device rapidly increases as fluid therein is warmed by body heat, and the device requires refrigeration.

The aforementioned shortcomings speak to the need for a small, lightweight, cost-effective device wherein cool temperature of a surface of the device is maintained over time to promote heat transfer from a body. In view of this, it is beneficial to have a cooling therapy device and method involving a thermally conductive surface. Temperature of the surface may be regulated by one or more airflow pathways. Alternatively, or additionally, temperature of the surface may be regulated by a heat exchanger, thermoelectric module, some combination thereof, or the like. An exemplary embodiment of the present invention is adapted to extract body heat from a palm of a user without requirements for liquid and/or vacuums.

According to the present invention in one aspect, an exemplary cooling therapy device comprises a rigid, thermally conductive surface. The rigid, thermally conductive surface may comprise lightweight, relatively inexpensive material. By way of example and not limitation, a portion of the thermally conductive surface may comprise aluminum, and/or any number of other lightweight, relatively inexpensive metals, including but not limited to combinations thereof. It will be apparent to one of ordinary skill in the art that any number of different rigid, thermally conductive materials, including but not limited to combinations thereof, may be employed without departing from the scope of the present invention.

An exemplary device of the present invention may be sized appropriately for contact with one or both palms and fingers of a user. It will be apparent to one of ordinary skill in the art that exemplary embodiments may be formed in different shapes and sizes suitable for contact with a user's palm(s). It will also be apparent to one of ordinary skill in the art that finger contact is not required to permit body heat transfer from a user.

The exemplary device may include a battery pack power supply positioned between structural brackets inside of the rigid, thermally conductive outer enclosure surface. The battery pack may comprise one or more rechargeable batteries regulated by an exemplary battery management system (“BMS”). An assembly for securing the battery pack and BMS may include through bolts, fasteners, fastener channels, and the like. The battery pack may be adapted to satisfy power requirements for one or more fans, heat exchangers, control units, thermoelectric modules, some combination thereof, or the like. It will be apparent to one of ordinary skill in the art that there are different methods and/or materials available for regulating temperature of the thermally conductive surface without departing from the scope of the present invention.

According to the present invention in another aspect, an exemplary cooling therapy device comprises a thermally conductive outer enclosure having a plurality of ambient air inlets and cooled air outlets adapted to permit one or more fans to circulate air within the device for cooling thereof. A user may place one or both of the user's palms on a portion of the outer enclosure of the device to contact the cold surface and/or encounter the cool air to thereby modify the body temperature of the user. Various other exemplary embodiments may be positioned on/within a handle, such as the handle of an aerobic or anaerobic exercise device.

It will be apparent to one of ordinary skill in the art that exemplary embodiments of the present invention provide a number of different advantages. Exemplary embodiments decrease internal heat accumulated in body tissues such as to, by way of example and not limitation, promote muscle strength, endurance, performance, and the like. An exemplary embodiment of the present invention is useful for promoting longer periods of high intensity exercise for a user, which may result in part from a user experiencing less bodily inflammation due to lower body temperature than would occur without the use of the invention.

The present invention provides a number of improvements over known devices for extracting/lowering body heat. By way of example and not limitation, the present invention is preferably small, lightweight, and cost effective, making the therapy device accessible to more users. The present invention is simple to use and simple to maintain. Operating the present invention is as simple as turning the device on and holding it in one's hand(s). Maintaining the invention may involve recharging and/or replacing one or more batteries thereof as said batteries drop below a minimum threshold state of charge.

### BRIEF DESCRIPTION OF THE DRAWINGS

Novel features and advantages of the present invention, in addition to those expressly mentioned herein, will become apparent to those skilled in the art from a reading of the following detailed description in conjunction with the accompanying drawings. The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 illustrates a top plan view of an exemplary cooling therapy device of the present invention;

FIG. 2 illustrates a perspective view of the exemplary cooling therapy device of FIG. 1;

FIG. 3 illustrates an exploded perspective view of a portion of the exemplary cooling therapy device of FIG. 1;

FIG. 4 illustrates a cross-section view of an exemplary power module of the exemplary cooling therapy device of FIG. 1;

FIG. 5 illustrates another perspective view of the exemplary cooling therapy device of FIG. 1;

FIG. 6 illustrates an exercise apparatus grip handle adapted to receive another exemplary palm cooling device of the present invention;

FIG. 7 illustrates another top plan view of the exemplary cooling therapy device of FIG. 1;

FIG. 8 illustrates an exemplary diagrammatic view of a high-level schematic electronics layout for the present invention.

### DETAILED DESCRIPTION OF EXEMPLARY EMBODIMENT(S)

Various embodiments of the present invention will now be described in detail with reference to the accompanying drawings. In the following description, specific details such as detailed configuration and components are merely provided to assist the overall understanding of these embodiments of the present invention. Therefore, it should be apparent to those skilled in the art that various changes and modifications of the embodiments described herein can be made without departing from the scope and spirit of the present invention. In addition, descriptions of well-known functions and constructions are omitted for clarity and conciseness.

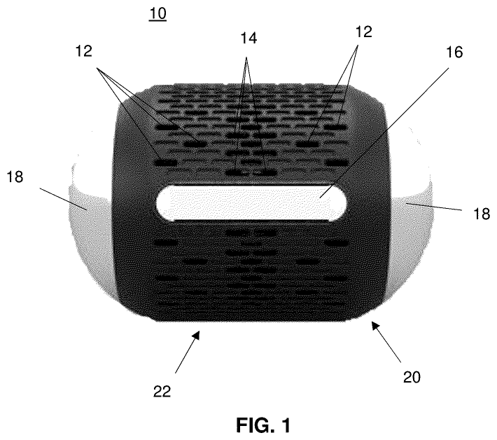

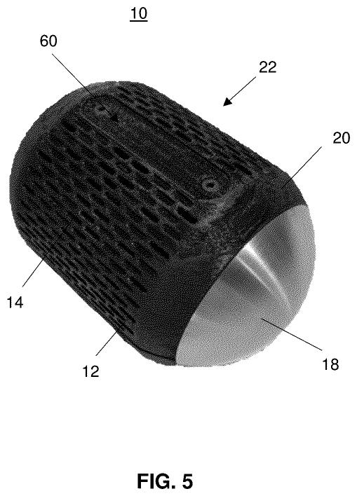

Referring now to FIGS. 1-5 and 7, an exemplary cooling therapy device 10 is shown. This particular embodiment includes a thermally conductive outer enclosure 20 configured with a plurality of ambient air inlets 12 and cool air outlets 14 adapted to permit one or more fans 26 to circulate air within the device 10 for cooling thereof. In this particular embodiment, a user may place one or both of the user's palms on a portion 22 of the outer enclosure 20 to modify the body temperature of the user. Specifically, as high volumes of blood flow through the user's palms positioned on the device 10, the user's body temperature may be reduced. The reduction of the user's body temperature may promote, for example, muscle strength, endurance, performance, and the like, such as for continued high intensity exercise for an additional period of time as desired by the user. Without body temperature cooling, accumulation of internal heat in user muscle tissue may adversely affect muscle strength, endurance, performance, and the like, due to such effects as inflammation in the body or muscle fatigue. The outer enclosure 20 may preferably comprise lightweight, relatively inexpensive, rigid material. The thickness of the outer enclosure 20 may be consistent with optimizing volumetric heat capacity.

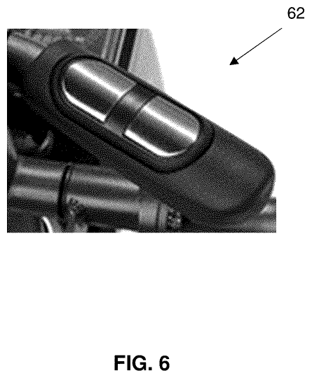

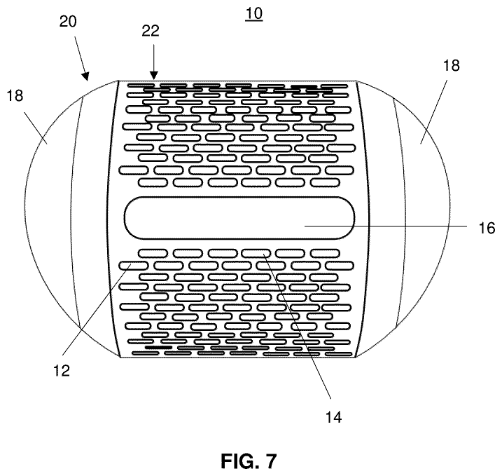

Referring to FIGS. 6-7, the exemplary cooling device 10 may be positioned on or made an integral part of a handle 62, such as the handle of an aerobic or anaerobic exercise device. It will be apparent to one of ordinary skill in the art that an exemplary cooling therapy device may be positioned on different exercise devices, such as aerobic (e.g., stationary bikes, elliptical machines, rowing machines, treadmills, and the like) and/or anaerobic exercise equipment (e.g., Olympic bars, curl bars, dumbbells, and the like). An exemplary cooling therapy device of the present invention may specifically be positioned on (or formed as a part of) a handle, grip, monitor, some combination thereof, or the like of said exercise equipment/apparatus in a location where the user may readily grip the portion having the invention thereon or therein, to promote heat transfer from a user's palm during an exercise (e.g., to promote improved performance). In this embodiment, the invention may be comprised of the same components as in the stand-alone embodiment, except it would be configured to fit in or fit on a portion of an exercise apparatus to be used by a user while exercising on or with the exercise apparatus.

Referring again to FIGS. 1-5 and 7, a user may position the palm(s) of one or both hands on a substantially thermally conductive mid-portion 22 of the exemplary cooling device 10. Faces 18 may be positioned on each side of the mid-portion 22, and the faces 18 and mid-portion 22 may collectively define an outer enclosure 20 of the device 10. The faces 18 may also be thermally conductive (e.g., comprise aluminum casings). The outer enclosure 20 may also include a label receptacle 60 adapted to receive a label plate or brand marking (e.g., 16), although such is not required. The label receptacle 60 may be configured to receive and secure a label plate 16 by way of one or more fasteners being secured through one or more corresponding apertures, or by way of an adhesive for example. A plurality of airflow outlets 14 may be centrally located across the surface of the mid-portion 22. A plurality of airflow inlets 12 may be located apart from the outlets 14 on each side of the mid-portion 22. It will be apparent to one of ordinary skill in the art that the specific shape, location and number of the various airflow inlets 12 and outlets 14 may be varied without departing from the scope of the present invention. It will also be apparent to one of ordinary skill in the art that an exemplary device may be defined by different shapes and/or sizes without departing from the scope of the present invention.

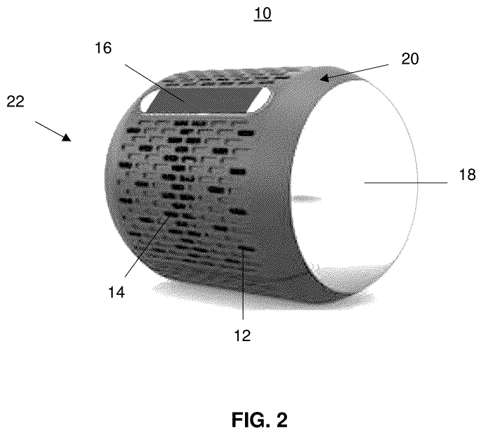

Referring specifically to FIGS. 2-4, a control module receptacle 32 may permit various objects within an interior 23 of the device 10 to be secured therein. A pin heat exchanger 36 and control circuitry 38 of the device 10 may be positioned within or proximate to the control module receptacle 32 (e.g., by way of fasteners 34). In the embodiment shown, the pin heat exchanger 36 and control circuitry 38 is secured between the control module receptacle 32 and a side attachment apparatus 40. The side attachment apparatus 40 shown is configured to immobilize one or more interior components (e.g., 36, 38) proximate to an aluminum casing face 18. Here, an interior frame of the side attachment apparatus 40 is configured to receive and secure the control circuitry 38. Operation of the various electronic components of the device, including a fan 26, may be regulated by the control circuitry 38. The control circuitry 38 may specifically dictate fan 26 speed, duration of fan 26 activity, heat exchanger 36 activity, thermoelectric module activity (if applicable), some combination thereof, or the like. A user may interact with the control circuitry 38 by way of a remote, control board on the device 10 exterior 20, some combination thereof, or the like. As a specific, non-limiting example, a user may select from within a predetermined temperature range (sufficient for safely extracting body heat) a specific operating temperature at the outer enclosure 20.

A user may also engage electronic controls of the device to dictate on and off status of the device. An exemplary device may be configured to operate no longer than a maximum amount of time during a single cooling therapy session in order to reduce or prevent the lowering of core body temperatures too low. Certain exemplary devices may include an electronic interface for communicating temperature (e.g., measured by a temperature sensor positioned in the device interior), state of charge, other diagnostic information, some combination thereof, or the like to a user. The temperature sensor may monitor temperature of the device when the device is in use, and operation of the device may be controlled at least in part based on the temperature readings of the temperature sensor.

The aforementioned interface may be engaged by a user to control certain therapy session parameters. Certain exemplary devices may be configured with an internal processor, or may be in electronic communication with an external processor to promote temperature regulation and the communication of device information. It will be apparent to one of ordinary skill in the art that there are a number of different methods/techniques available for regulating interior components of an exemplary device without necessarily departing from the scope of the present invention.

An attachment apparatus 28 may permit securement of the fan 26 proximate to the control module receptacle 32 within an interior 23 of the device 10. Specifically, the fan 26 may be secured by way of one or more fasteners 24 being positioned in apertures of the attachment apparatus 28. The control module receptacle 32 and side attachment apparatus 40 may each be secured to inner walls (not shown) of the device 10 outer enclosure 20, such as by clips, fasteners, some combination thereof, or the like. It will be apparent to one of ordinary skill in the art that exemplary components may be secured within an exemplary device by way of different connection methods or techniques without departing from the scope of the present invention.

In the embodiment shown, movement of the fan 26 causes ambient air to be drawn into the device 10 interior through air inlets 12. Said air may be directed from the inlets 12 to the heat exchanger 36 by way of a number of inbound air flow channels. Here, the heat exchanger 36 is configured to decrease the temperature of said air. Conductive surfaces of the heat exchanger 36 may be cooled by a number of different thermoelectric modules, convection-based cooling techniques, cross-fluid heat exchange, some combination thereof, or the like. A thermoelectric module may regulate temperature(s) within the heat exchanger 36, and may be controlled by the control circuitry 38. A thermoelectric module may include a Peltier module. The various electronic components of the device 10 may be powered by a power module 30. The power module 30 may comprise a battery, power board, some combination thereof, or the like. It will also be apparent to one of ordinary skill in the art that different air temperature modification techniques involving a heat exchanger, heat sink, thermoelectric module, fluid crossflow heat transfer device, some combination thereof, or the like (individually or collectively, “air temperature modification devices”) may be employed for decreasing air temperature without departing from the scope of the present invention.

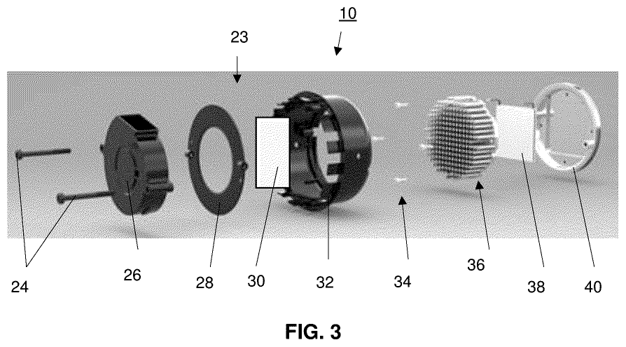

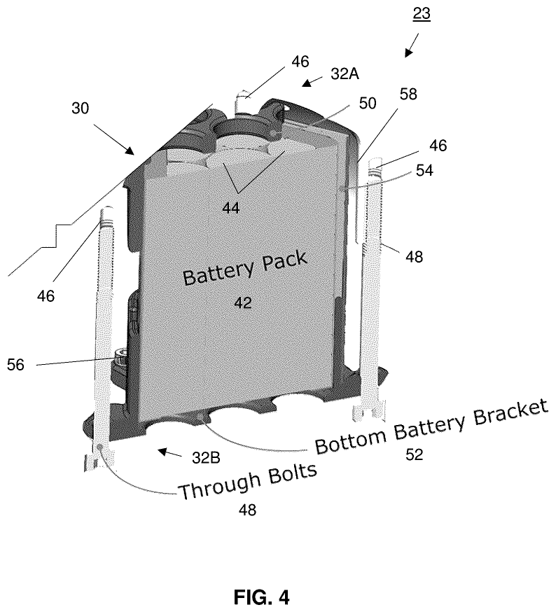

Referring now specifically to FIGS. 3-4, an exemplary power module 30 positioned in an interior 23 of the device 10 comprises a rechargeable battery pack 42. Electronic communication between the battery pack 42 and each of various electronic components (e.g., 26, 36, 38) of the device 10 may be established by a wire. A charging port of the battery pack 42 may permit a user to connect a charging device to the battery pack 42 to recharge one or more batteries 44 of the battery pack 42. It will be apparent to one of ordinary skill in the art, however, that rechargeable batteries are not necessarily required in certain other embodiments. Electronic component (e.g., 26, 36, 38) power requirements may be satisfied by a number of different power sources without departing from the scope of the present invention. Batteries, whether rechargeable or single use, are not necessarily required to satisfy power requirements for the present invention. Various battery technologies from alkaline to lithium ion and many more varieties may be suitable for the present invention.

In this particular embodiment, the battery pack 42 is secured between a top battery bracket 50 and a bottom battery bracket 52 within the control module receptacle 32 of the interior 23. It will be apparent to one of ordinary skill in the art that the particular sizes and shapes of battery brackets shown are merely illustrative, and a number of different materials and/or mechanisms may be employed to secure an exemplary battery pack within the interior without departing from the scope of the present invention. The battery pack 42 may comprise a number of batteries 44. The battery pack 42 may be regulated by an exemplary BMS 54. The BMS 54 may be configured to ensure the batteries 44 operate within a safe operating area, monitor voltage, monitor battery temperature, monitor current, monitor state of charge, some combination thereof, or the like. A portion 58 of the power module 30 may be configured to receive and secure the BMS 54.

The top 50 and bottom 52 battery brackets may each be sized to maintain and restrict movement of the battery pack 42 within the interior 23 of the device 10. The bottom battery bracket 52 may be secured (e.g., by way of one or more fasteners 56) to a lower portion 32B of the control module receptacle 32. Specifically, the one or more fasteners 56 may be positioned through an aperture of the bottom battery bracket 52 and into a respective fastener channel of the lower portion 32B of the control module receptacle 32. The bottom battery bracket 52 may further be secured within the interior 23 of the device 10 by positioning each of one or more through bolts 48 through an aperture of the bottom battery bracket 52 and into a respective fastener channel 46 at an upper portion 32A of the control module receptacle 32. A user may be permitted to loosen each of the one or more through bolts 48 to disengage said bolts 48 from the fastener channels 46 (e.g., to replace a battery pack 42).

The top battery bracket 50 may be secured to the upper portion 32A of the control module receptacle 32 by positioning each of one or more pegs, fasteners, or the like in each of a top battery bracket aperture (not shown) and an upper portion 32A channel (not shown). It will be apparent to one of ordinary skill in the art that the specific configurations for securing interior components of the device illustrated and described herein are in no way intended to be exhaustive of the scope of the present invention. Any number of different materials and/or mechanisms for securing electronic components within a device interior may be employed without departing form the scope of the present invention.

Referring specifically to FIGS. 2-3, as said air flows across said conductive surfaces of the heat exchanger 36, the temperature of said air may be significantly reduced. The reduced-temperature air may then be directed to air outlets 14 by way of a number of outbound air flow channels. The reduced temperature air may then be discharged through said outlets 14, and may thereafter contact a user's palms to reduce body temperature of the user. The reduced temperature air may also cool a conductive surface of the device 10 exterior 20 to further contribute to reducing body temperature of the user (e.g., byway of heat removal from the user's palm(s)). As a non-limiting example, a user may place a first hand on a first side of the device 10 exterior 20, and a second hand on a second side of the device 10 exterior 20 such that both palms of the user are directly exposed to cold air being discharged from outlets 14, and a cool surface of the mid-portion 22. It will be apparent to one of ordinary skill in the art that the specific arrangement of interior and exterior components shown is merely illustrative, and variations may be made without necessarily departing from the scope of the present invention.

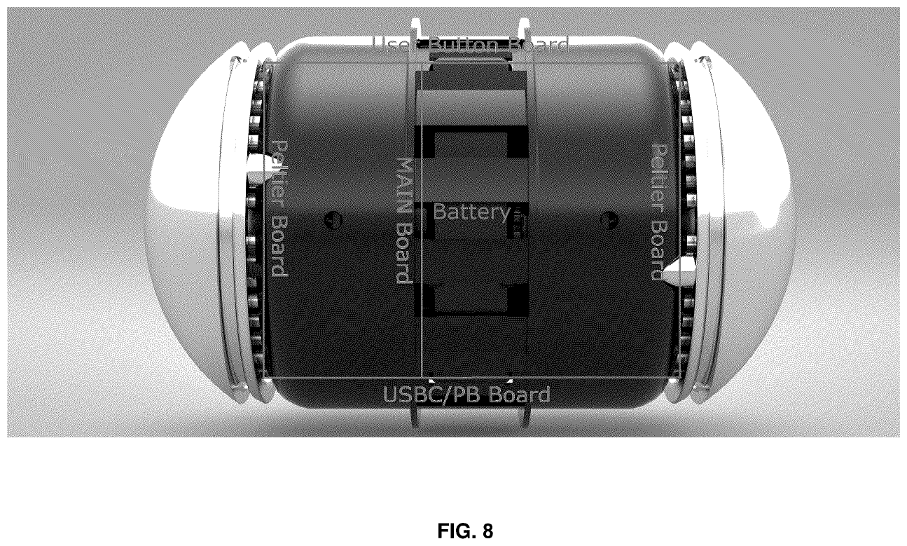

Referring now to FIG. 8, an example high-level electrical schematic diagram is shown overlayed on an exemplary embodiment of the cooling therapy device of the present invention. An electronics board for operation button controls is indicated, as is a main processor control board, a Peltier control board, and a USBC/PB Board, powered by the enclosed battery or battery pack within the device. The invention electronics may be controlled by a microcontroller which may be in electronic communication with: an LED driver circuit which may be incorporated for operational signals; a battery charging circuit and battery connector; a power push button controller circuit; a power circuit; one or more Peltier power circuit(s), a Peltier engine feedback circuit, and a Peltier current feedback circuit.

Any embodiment of the present invention may include any of the features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to the described invention. Many of those variations and modifications will provide the same result and fall within the spirit of the claimed invention. It will also be apparent to one of ordinary skill in the art that exemplary embodiments of the present invention are not necessarily intended to be limited to use with heat extraction for promoting muscle strength, endurance, performance, and the like. Exemplary embodiments of the present invention may also be useful for decreasing core body temperature to, e.g., address a fever. It is the intention, therefore, to limit the invention only as indicated by the scope of the claims.

Certain operations described herein may be performed by one or more electronic devices. Each electronic device may comprise one or more processors, electronic storage devices, executable software instructions, and the like configured to perform the operations described herein. The electronic devices may be general purpose computers or specialized computing device. The electronic devices may comprise personal computers, smartphone, tablets, databases, servers, processors, or the like, internal or external to the device, and when internal may be small or miniature size. The electronic connections and transmissions described herein may be accomplished by wired or wireless means.

What is claimed is:

1. A cooling therapy device, comprising:
a thermally conductive outer enclosure having at least one ambient air inlet
and at least one cool air outlet;
an interior portion, comprising:
a power supply secured within said outer enclosure;
a heat exchanger secured within said outer enclosure; and
wherein said heat exchanger is configured to generate a cooler temperature
at said outer enclosure than the temperature of said ambient air, and
wherein said device is configured to be hand-held.

2. The device of claim 1, wherein said device, at said outer enclosure, is configured to reach temperatures low enough to cool body temperature of a user for a limited period of time.

3. The device of claim 2, wherein said outer enclosure comprises aluminum.

4. The device of claim 1, further comprising a plurality of cool air outlets on an outer surface of said outer enclosure.

5. The device of claim 1, wherein said device further comprises a battery pack secured between brackets within a receptacle.

6. The device of claim 1, wherein said device is secured within and made part of a grip portion of an exercise machine.

7. A cooling therapy device, comprising:
An outer enclosure configured to be hand-held and having
a plurality of ambient air inlets and a plurality of air outlets, configured to discharge cool air on glabrous skin of a user;
an interior portion comprising:
a fan;
an air temperature modification module;
control circuitry;
wherein said fan is configured to cause ambient air to be drawn in through one or more of said air inlets and directed to said air temperature modification module;
wherein said air temperature modification module is configured to reduce temperature of said ambient air resulting in cooler air; and
wherein said interior portion is capable of directing said cooler air to said air outlets to be discharged therefrom.

8. The device of claim 7, wherein said device is secured within and made a part of a portion of an exercise device.

9. The device of claim 8, wherein said device is secured in a handle of an aerobic exercise device.

10. The device of claim 9, wherein said aerobic exercise device is an elliptical machine.

11. The device of claim 9, wherein said aerobic exercise device is a stationary bicycle.

12. The device of claim 7, wherein said outer enclosure is comprised of a first substantially hemisphere portion secured to a first end of a substantially cylindrical portion and a second substantially hemisphere portion is secured to a second end of said cylindrical portion.

13. The device of claim 7, wherein said temperature modification module is comprised of a peltier module.

14. The device of claim 12, wherein said plurality of ambient air inlets and said plurality of cooler air outlets are positioned across a circumference of said substantially cylindrical exterior portion.

15. The device of claim 7, wherein said outer enclosure comprises aluminum.

16. The device of claim 7, further comprising a control module receptacle, wherein said control module receptacle is configured to secure one or more interior portion components of said device.

17. A cooling therapy device, comprising:
an exterior enclosure portion configured to be hand-held, having a plurality of ambient air inlets and a plurality of cool air outlets, configured to discharge cool air on glabrous skin of a user;
an interior portion comprising:
at least one fan;
an air temperature modification module;
a power supply and control circuitry, adapted to drive said at least one fan and said air temperature modification module;
wherein said at least one fan is configured to be capable of causing air to be drawn in through said air inlets and directed to said air temperature modification module;
wherein said air temperature modification module is configured to reduce temperature of said ambient air drawn inside said device and to expel cooler air than said ambient air temperature through said outlets.

18. The invention of claim 17, wherein said temperature modification module comprises a peltier module.

19. The invention of claim 17, further comprising a temperature sensor in said device for monitoring a temperature during use of said device and controlling operation of the device at least in part from said temperature reading.
